(19) United States
(12) Patent Application Publication
(10) Pub. No.: US 2011/0269171 A1
(43) Pub. Date: Nov. 3, 2011

(54) METHODS AND MATERIALS FOR USING NKAP POLYPEPTIDE EXPRESSION LEVELS OR PATTERNS TO DETECT OR ASSESS CANCER

(76) Inventors: Virginia M. Shapiro, Rochester, MN (US); Michael J. Shapiro, Rochester, MN (US); Rhianna S. Sundsbak, Rochester, MN (US)

(21) Appl. No.: 13/089,998

(22) Filed: Apr. 19, 2011

Related U.S. Application Data
(60) Provisional application No. 61/325,726, filed on Apr. 19, 2010.

(51) Int. Cl. C12Q 1/02

(52) U.S. Cl. 435/29

(57) ABSTRACT

This document relates to methods and materials involved in detecting or assessing cancer (e.g., ovarian cancer). For example, methods and materials involved in using NKAP polypeptide expression levels and patterns to diagnose cancer, assess cancer survival, and assess cancer treatment options are provided.

### CROSS-REFERENCE TO RELATED APPLICATIONS

This application claims the benefit of U.S. Provisional Application Ser. No. 61/325,726, filed Apr. 19, 2010. The disclosure of the prior application is considered part of (and is incorporated by reference in) the disclosure of this application.

### STATEMENT AS TO FEDERALLY SPONSORED RESEARCH

This invention was made with government support under grant number AI069031 awarded by National Institute of Allergy and Infectious Disease. The government has certain rights in the invention.

### BACKGROUND

1. Technical Field

This document relates to methods and materials involved in detecting or assessing cancer (e.g., ovarian cancer). For example, this document relates to methods and materials involved in using NKAP polypeptide expression levels or expression patterns to diagnose cancer, assess cancer survival, and assess cancer treatment options.

2. Background Information

There are many types of cancers that affect humans. For example, pancreatic cancer remains one of the most lethal human cancers, with a 5-year survival rate of only 5%. Ovarian cancer is the most lethal of the gynecologic malignancies, largely due to it being diagnosed when the disease is already advanced and its propensity to develop chemoresistance.

### SUMMARY

This document provides methods and materials involved in detecting or assessing cancer (e.g., ovarian cancer). For example, this document provides methods and materials involved in using NKAP polypeptide expression levels or expression patterns to diagnose cancer, assess cancer survival, and assess cancer treatment options.

This document is based, in part, on the discovery that reduced levels of NKAP polypeptide expression and/or cytoplasmic localization of NKAP polypeptide expression in tissue suspected to be cancerous can used to identify that tissue as cancerous. For example, cancers such as pancreatic cancer, ovarian cancer, T cell acute leukemias (e.g., T-cell acute lymphoblastic leukemia), and glioblastomas can be identified based on a reduced level of NKAP polypeptide expression in a tissue sample used to identify the presence of cancer. In the case of pancreatic cancer, for example, a reduced level of NKAP polypeptide expression in a pancreatic tissue sample can indicate that the pancreatic tissue is cancerous.

This document also is based, in part, on the discovery that reduced levels of NKAP polypeptide expression and/or cytoplasmically expressed NKAP polypeptides in cancer tissue (e.g., ovarian cancer tissue) can indicate that the patient has an increased risk of having accelerated relapse and a shorter survival time. For example, stage 4 ovarian cancer patients can be identified as and informed about having an accelerated relapse time and/or a short survival time based on the presence of ovarian cancer cells that express a reduced level of NKAP polypeptides and/or that cytoplasmically express NKAP polypeptides.

In some cases, the methods and materials provided herein can be used to identify cancer patients who are unlikely to respond to cancer treatments such as those that include the use of gamma-secretase inhibitors (e.g., BMS-708163, GSI-136, and GSI-953). For example, cancer patients can be identified as and informed about having reduced chance of responding positively to gamma-secretase inhibitor treatments based on the presence of cancer cells that express a reduced level of NKAP polypeptides and/or that have a cytoplasmic NKAP expression pattern.

In general, one aspect of this document features a method for identifying a mammal as having cancer. The method comprises, or consists essentially of, (a) detecting the presence of cells having aberrant NKAP polypeptide expression within a tissue sample from the mammal, and (b) classifying the mammal as having the cancer based at least in part of the presence of the cells. The mammal can be a human. The cells can be ovarian, pancreatic, neuronal, immune, or prostate cells. The aberrant NKAP polypeptide expression can be reduced NKAP polypeptide expression. The aberrant NKAP polypeptide expression can be cytoplasmic expression of the NKAP polypeptide.

In another aspect, this document features a method for assessing a cancer patient's risk for cancer relapse. The method comprises, or consists essentially of, (a) determining whether or not a tissue sample from the patient comprises cells having aberrant NKAP polypeptide expression, (b) classifying the patient as having an increased likelihood for cancer relapse if the cells are present within the sample, and (c) classifying the patient as not having an increased likelihood for cancer relapse if the cells are absent from the sample. The patient can be a human patient. The patient can be a stage 4 ovarian cancer patient. The cells can be ovarian cells. The aberrant NKAP polypeptide expression can be reduced NKAP polypeptide expression. The aberrant NKAP polypeptide expression can be cytoplasmic expression of the NKAP polypeptide.

In another aspect, this document features a method for assessing a cancer patient's likelihood for prolonged cancer survival, wherein the method comprises, or consists essentially of, (a) determining whether or not a tissue sample from the patient comprises cells having aberrant NKAP polypeptide expression, (b) classifying the patient as having a reduced likelihood for prolonged cancer survival if the cells are present within the sample, and (c) classifying the patient as having an increased likelihood for prolonged cancer survival if the cells are absent from the sample. The patient can be a human patient. The patient can be a stage 4 ovarian cancer patient. The cells can be ovarian cells. The aberrant NKAP polypeptide expression can be reduced NKAP polypeptide expression. The aberrant NKAP polypeptide expression can be cytoplasmic expression of the NKAP polypeptide.

In another aspect, this document features a method for assessing a cancer patient's likelihood for responding to a gamma-secretase inhibitor cancer treatment, wherein the method comprises, or consists essentially of, (a) determining whether or not a tissue sample from the patient comprises cells having aberrant NKAP polypeptide expression, (b) classifying the patient as having a reduced likelihood of responding to the gamma-secretase inhibitor cancer treatment if the cells are present within the sample, and (c) classifying the patient as being likely to respond to the gamma-secretase inhibitor cancer treatment if the cells are absent from the sample. The cancer patient can be a human patient. The cancer patient can be a patient diagnosed with ovarian cancer, pancreatic cancer, leukemia, glioblastoma, T cell lymphomas, colon adenocarcinomas, or prostate cancer. The cells can be ovarian, pancreatic, neuronal, immune, or prostate cells. The aberrant NKAP polypeptide expression can be reduced NKAP polypeptide expression. The aberrant NKAP polypeptide expression can be cytoplasmic expression of the NKAP polypeptide.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 contains photographs of ovarian cancer tissue stained with anti-NKAP antibodies and hematoxylin. Panel A shows normal levels of NKAP expression with normal nuclear localization of NKAP. Panel B shows low/negative NKAP expression with greater hematoxylin staining than NKAP staining Panel C shows NKAP expression with aberrant localization—the cytoplasmic staining is equal to or greater than the nuclear staining.

FIG. 2 is a table presenting the number of patients with serous ovarian carcinoma at the indicated stage with normal NKAP expression or aberrant NKAP expression (e.g., low/negative NKAP expression and/or aberrant NKAP localization). Alterations in NKAP expression resulted in a worse outcome for ovarian cancer patients. For patients with serous carcinomas with optimal debulking after surgery and normal nuclear expression of NKAP, 15/33 (45.5%) of patients had relapsed, and 13/33 (38.2%) of patients had died. For patients with serous carcinomas with optimal debulking after surgery and aberrant expression of NKAP (either low/negative or mislocalized to the cytoplasm), 9/11 (81.8%) of patients had relapsed, and 8/12 (66.7%) of patients had died. Therefore, examination of NKAP expression can predict patients for whom current treatments are less effective and for whom alternative therapies may be considered.

FIG. 3 is a graph plotting the relapse free survival data for serous ovarian cancer patients with normal NKAP expression (normal) or aberrant NKAP expression (aberrant). The NKAP expression group includes those patients with low/negative NKAP expression and/or aberrant cytoplasmic NKAP localization. P=0.0327 via the Gehan-Breslow-Wilcoxon Test. The median relapse free survival for NKAP high nuclear (normal) was 33 months, while the median relapse free survival for NKAP low/cyto (aberrant) was 16 months. The ratio was 2.063, and the 95% CI of ratio was 1.625 to 2.500. These results indicate that the loss of normal NKAP expression has negative implications in relapse of serous ovarian cancer.

FIG. 4 is a graph plotting the overall survival data for serous ovarian cancer patients with normal NKAP expression (normal) or aberrant NKAP expression (aberrant). The NKAP expression group includes those patients with low/negative NKAP expression and/or aberrant cytoplasmic NKAP localization. P=0.2134 via the Gehan-Breslow-Wilcoxon Test. The median overall survival for NKAP high nuclear (normal) was 55 months, while the median overall survival for NKAP low/cyto (aberrant) was 41 months. The ratio was 1.362.

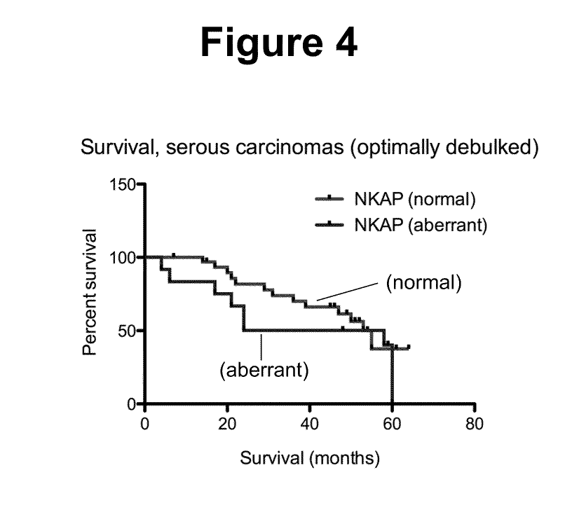

FIG. 5 is a graph plotting the overall survival data for stage 4 ovarian cancer patients with normal NKAP expression (normal) or aberrant NKAP expression (aberrant). The NKAP expression group includes those patients with low/negative NKAP expression and/or aberrant cytoplasmic NKAP localization. P=<0.0001 via the Gehan-Breslow-Wilcoxon Test. The median overall survival (n=15) for NKAP high nuclear (normal) was 31.5 months, while the median overall survival for NKAP low/cyto (aberrant) was 3 months.

FIG. 6 is a Kaplan Meier survival curve plotting time to death for patients with ovarian serous cancer with optimal debulking with normal NKAP expression (“NKAP1”), low/negative NKAP expression (“NKAP2”), or cytoplasmicially mislocalized NKAP expression (“NKAP3”). This shows that mislocalization of NKAP is associated with poor outcome with respect to overall survival, with HR=2.19, 95% CI: 1.18-4.05, p=0.01.

FIG. 7 is a Kaplan Meier survival curve plotting time to recurrence for patients with ovarian serous cancer with optimal debulking with normal NKAP expression (“NKAP1”), low/negative NKAP expression (“NKAP2”), or cytoplasmically mislocalized NKAP expression (“NKAP3”). This shows that NKAP mislocalization is associated with an accelerated time to recurrence, with HR=3.59, 95% CI: 1.75-7.35, p=0.0005.

FIG. 8 contains photographs of T cell acute leukemias stained with anti-NKAP antibodies and hematoxylin. Panel A shows negative NKAP expression. Panel B shows weak NKAP expression. Panel C shows strong levels of NKAP expression.

FIG. 9 is a graph plotting the relapse free survival data for T cell acute leukemia patients with normal NKAP expression (normal) or weak/negative NKAP expression (weak/negative). Of 14 patients who entered remission, 3/10 (30%) of patients with NKAP expression relapsed, while 0/4 patients with low/negative NKAP expression relapsed.

FIG. 10 is a graph plotting the overall survival data for T cell acute leukemia patients with normal NKAP expression (normal) or weak/negative NKAP expression (weak/negative). 5/12 (42%) of patients with strong/moderate NKAP expression died, while ⅕ (20%) of patients with weak/negative NKAP staining died.

FIG. 11 contains photographs of pancreatic adenocarcinomas stained with anti-NKAP antibodies. Of the pancreatic adenocarcinomas from 102 patients examined, 23/102 (22.5%) had normal nuclear expression of NKAP (top panel), 59/102 (57.8%) had low/negative NKAP expression (middle panel), and 20/102 (19.6%) had NKAP mislocalized to the cytoplasm (bottom panel).

FIG. 12 contains photographs of glioblastoma xenografts stained with anti-NKAP antibodies and hematoxylin. Panel A shows NKAP and hematoxylin co-staining of normal white matter. Panel B shows weak NKAP expression. Panel C shows strong levels of NKAP expression. 6/24 (25%) glioblastoma xenografts exhibited low/negative NKAP expression, while 18/24 (75%) glioblastoma xenografts exhibited high NKAP expression.

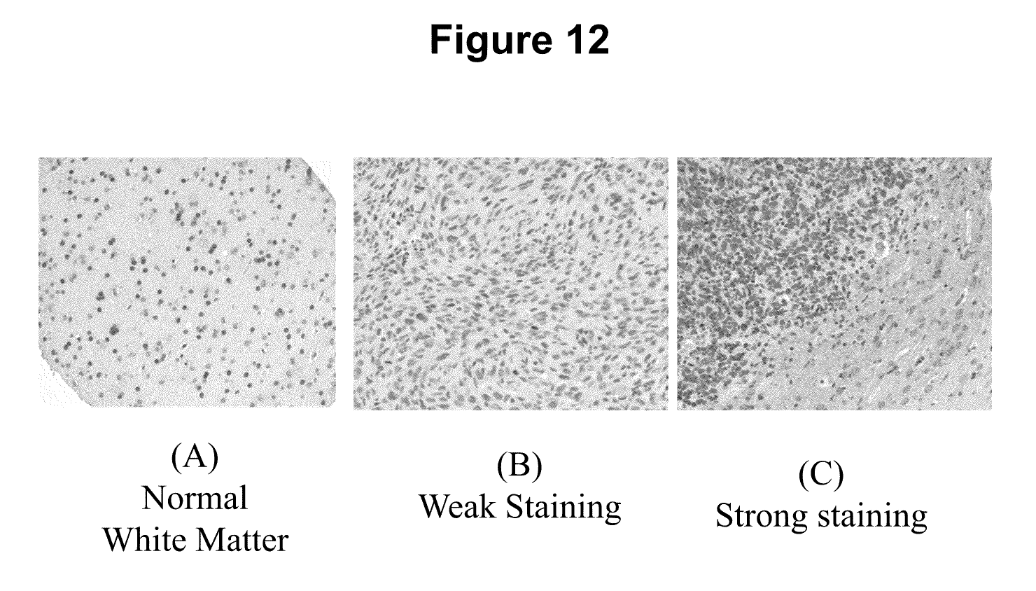

FIG. 13. Pdx1-cre NKAP cKO mice develop mucinous cysts at 12 months (left) and 14 months (right).

FIG. 14. Examination of mucinous cyst from 12 month old pdx1-cre NKAP cKO mouse. The mucinous cyst from a 12 month old pdx1-cre NKAP cKO mouse was fixed in formalin, embedded, and examined by IHC for the antibodies/stains as denoted in the figure.

FIG. 15. Examination of the pancreas with localized pancreatic adenocarcinoma from a seven month old pdx1-cre LSL-K-Ras G12D/NKAP cKO mouse. The pancreas from a seven month old pdx1-cre LSL-K-Ras G12D/NKAP cKO mouse was fixed in formalin, embedded, and examined by IHC for the antibodies/stains as denoted in the figure.

FIG. 16 contains four graphs plotting overall survival for glioblastoma xenografts with wild-type or normal NKAP expression (WT or normal) or weak/negative NKAP expression (low/neg). RT and TMZ refer to treatments given the mice that parallel treatments used in therapy for glioblastoma patients. RT is radiation therapy. TMZ is temozolomide.

FIG. 17 is a graph plotting the overall survival data for glioblastoma patients with normal NKAP expression (normal) or low/negative NKAP expression (low/negative). Loss of NKAP expression did not affect overall survival.

FIG. 18 is a Haploview LD plot that shows the LD (linkage disequilibrium) present amongst the indicated 11 SNPs used to analyze the NKAP locus in the pancreatic adenocarcinoma GWAS (genome wide association study).

FIG. 19 is an Association LD plot of the 11 SNPs in the NKAP locus used in these studies. One SNP (rs7063278) had a p-value of 0.039 for females in the survival analysis. No other tested SNP had p-values <0.05 in any analysis. The upper dashed line refers to a p=0.05, and the lower dashed line refers to a p=0.10.

FIG. 20 is a Kaplan Meier survival curve for the pancreatic adenocarcinoma samples examined for NKAP expression by immunohistochemistry. Overall survival was examined separately in patients with pancreatic adenocarcinomas with either normal, low/negative, or cytoplasmic NKAP expression.

FIG. 21 is a Kaplan Meier survival curve for the pancreatic adenocarcinoma samples examined for NKAP expression by immunohistochemistry. Overall survival was examined separately in patients with pancreatic adenocarcinomas with either normal or aberrant (low/negative and/or cytoplasmic samples combined) NKAP expression.

FIG. 22 is a Kaplan Meier survival curve for PanINs (pre-cancerous pancreatic intraepithelial lesions) examined for NKAP expression by immunohistochemistry. Overall survival was examined separately in patients with PanINs with either normal, low/negative, or cytoplasmic NKAP expression.

FIG. 23 is a Kaplan Meier survival curve for PanINs examined for NKAP expression by immunohistochemistry. Overall survival was examined separately in patients with PanINs with either normal or aberrant (low/negative and/or cytoplasmic samples combined) NKAP expression.

### DETAILED DESCRIPTION

This document provides methods and materials involved in detecting or assessing cancer (e.g., ovarian cancer) in mammals. For example, this document provides methods and materials involved in using NKAP polypeptide levels to diagnose cancer, assess cancer survival (e.g., relapse-free survival), and assess cancer treatment options. The mammal can be any type of mammal such as a human, monkey, dog, cat, horse, cow, goat, mouse, or rat. The wild-type human NKAP polypeptide (GenBank GI No. 168229155) is a 415-amino acid, widely expressed nuclear protein that is highly conserved from fly to human (Chen et al., Biochem. Biophys. Res. Commun., 310:720-724 (2003); and (Pajerowski et al., Immunity, 30:696-707 (2009))). The NCBI reference mRNA sequence for human NKAP is NM_{—}024528, and the NCBI reference polypeptide sequence for human NKAP is NP_{—}078804.

As described herein, a mammal can be diagnosed as having cancer (e.g., pancreatic cancer, ovarian cancer, T cell acute leukemias such as T-cell acute lymphoblastic leukemia, and glioblastomas), as opposed to a benign condition, if it is determined that a tissue biopsy obtained to assess a mammal for cancer contains cells that express low levels of an NKAP polypeptide and/or NKAP polypeptides with a cytoplasmic localization. In such cases, the mammal can be classified as having cancer (e.g., cancer of the type being assessed based on the type of tissue biopsy) and can be treated for that type of cancer. Any cell type suspected of being cancerous can be isolated and evaluated for aberrant NKAP polypeptide expression as described herein. For example, ovarian tissue cells, pancreatic tissue cells, T cells, neurons, colon cells, and prostate cells can be isolated and evaluated for aberrant NKAP polypeptide expression. Any appropriate method can be used to assess cells for aberrant NKAP polypeptide expression. For example, immuno-based assays such as immunohistochemistry can be used to identify cells with aberrant NKAP polypeptide expression. In some case, nucleic acid-based assays such as PCR can be used to detect nucleic acid mutations that result in reduced levels of NKAP polypeptide expression or NKAP polypeptide expression with a cytoplasmic localization. In some cases, molecular assays can be used to assess NKAP nucleic acid copy number or the expression of surrogate markers of aberrant NKAP polypeptide expression (e.g., increased expression of Deltex1 polypeptides) to assess cells for aberrant NKAP polypeptide expression. As described herein, Deltex expression can be upregulated upon loss of NKAP. Deltex expression also can be upregulated by activating mutations in Notch. Thus, Deltex expression may not be a specific readout for loss of NKAP, but may be found where NKAP expression is altered.

In some cases, the methods and materials provided herein can be used to determine if a mammal with cancer has an increased risk of having accelerated relapse and/or a shorter survival time. For example, stage 4 ovarian cancer patients can be identified as and informed about having an accelerated relapse time and/or a short survival time based on the presence of ovarian cancer cells that express a reduced level of NKAP polypeptides and/or that express NKAP polypeptides with a cytoplasmic localization.

In some cases, the methods and materials provided herein can be used to identify a mammal as having cancer that is unlikely to respond to cancer treatments such as those that include the use of gamma-secretase inhibitors (e.g., BMS-708163, GSI-136, and GSI-953). For example, cancer patients can be identified as and informed about having reduced chance of responding positively to gamma-secretase inhibitor treatments based on the presence of cancer cells that express a reduced level of NKAP polypeptides and/or that express NKAP polypeptides with a cytoplasmic localization.

The invention will be further described in the following examples, which do not limit the scope of the invention described in the claims.

### EXAMPLES

### Example 1

### NKAP Expression and Ovarian Cancer

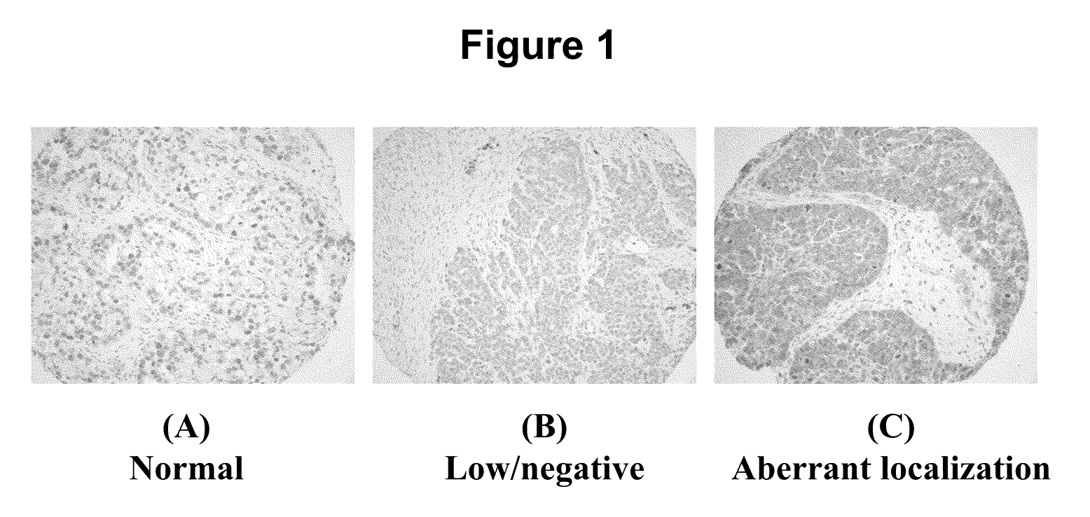

To examine NKAP expression in ovarian cancer, three tissue microarrays (TMA) were examined for NKAP polypeptide expression by immunohistochemistry (IHC), using an antisera developed against NKAP. Normally, NKAP is localized to the nucleus, and stains brightly in normal human ovarian epithelial cells and is similarly localized in ovarian tumors (FIG. 1, panel A). A subset of ovarian cancers had either very low/negative levels of NKAP expression (FIG. 1, panel B) or displayed aberrant localization of NKAP to the cytosol (FIG. 1, panel C). Overall, of the 87 patients examined, 23% of the ovarian cancers analyzed by IHC had aberrant NKAP expression (either low/negative or cytoplasmic localization). This initial dataset was too small for statistically significant multivariate analysis, but initial univariate analysis demonstrates that NKAP may be a new biomarker for ovarian cancer. Since the TMAs contained a variety of ovarian cancers by type, stage, and grade, and to ensure that the initial statistical comparison of outcome was between equivalent samples, the analysis was limited to only serous carcinomas with optimal debulking after surgery (45 out of 87 patient samples). Since NKAP is a transcriptional repressor that regulates gene expression in the nucleus, the aberrant localization of NKAP to the cytoplasm would result in the loss of NKAP function. Therefore, both low/negative expression and aberrant cytoplasmic localization would have the same consequence of eliminating NKAP function, and thus samples with either anomaly were counted together in a single group in this initial analysis (since separately the sample size was insufficient to allow for any statistical analyses).

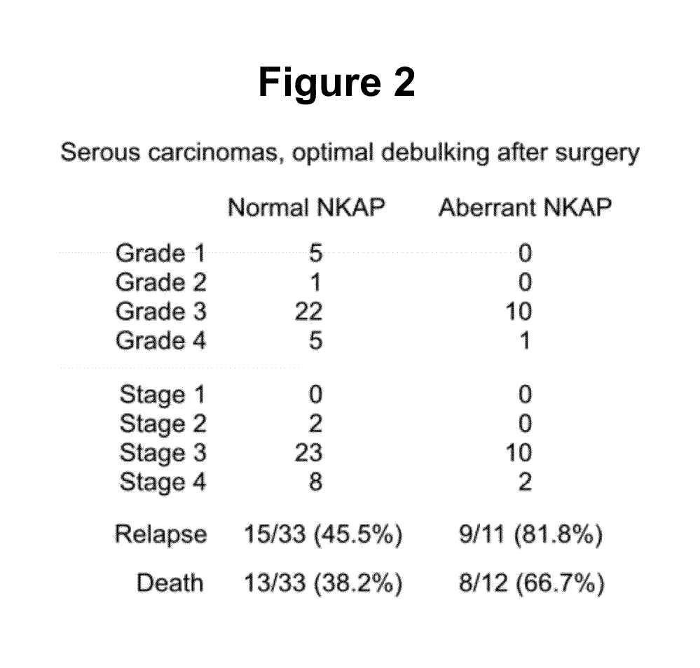

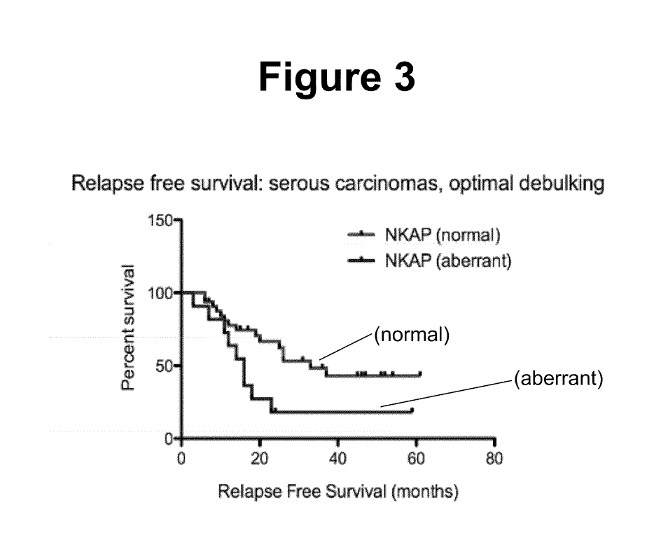

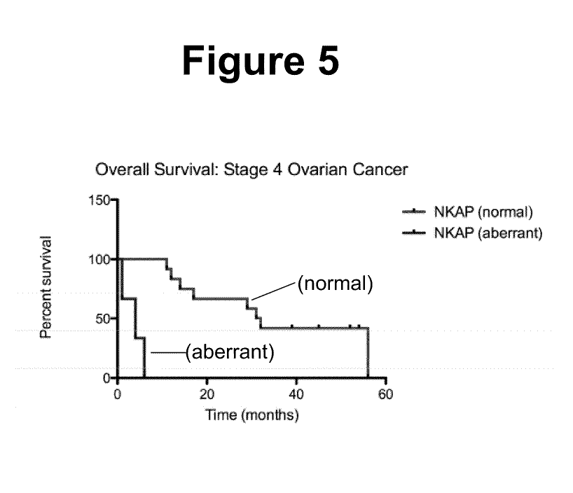

A comparison of the stage, grade, and outcome is listed in FIG. 2. Although similar across stage and grade, the outcomes for relapse and survival are different for serous carcinomas that express NKAP within the nucleus as compared to those with aberrant expression of NKAP. Overall, 45.5% ( 15/33) of patients with normal NKAP expression relapsed, as compared to 81.8% ( 9/11) with aberrant NKAP expression (one patient never went into remission, and was therefore not included in analysis of relapse free survival). In addition, 38.2% ( 13/33) of patients with normal NKAP expression had died, as compared to 66.7% ( 8/12) with aberrant NKAP expression. Kaplan-Meier analysis of relapse-free survival in this set of patients is shown in FIG. 3. The relapse-free survival for aberrant NKAP expressors was significantly different (p<0.02 by Log-rank Mantel-Cox test), with a median relapse free survival of 16 months as compared to 33 months for normal NKAP expressors (FIG. 3, hazard ratio of 2.0625). There is a trend towards differences in the overall survival, with median overall survival for normal NKAP expression of 55 months as compared to 41 months for aberrant NKAP expression, but it is not yet statistically significant. Strikingly, if the analysis is limited only to those patients with stage four disease (across all types of ovarian cancer), the median overall survival for those with aberrant NKAP expression is four months, with no patients ( 0/3) surviving beyond 6 months (FIG. 5). In comparison, stage four disease with normal NKAP expression had a median survival of 31.5 months, with 4/12 patients still alive (at 39, 45, 52 and 54 months) post-diagnosis. Although the sample size for stage four disease is small (15 total), it is statically significant (p<0.0001 using Log-rank Mantel-Cox analyses). This appears to be one of the strongest single-gene effects on outcome observed. Therefore, these results indicate that expression of NKAP is highly variable, and that loss of NKAP expression or mislocalization of NKAP to the cytoplasm is a new and significant prognostic indicator of outcome in ovarian cancer. In support of this, a screening of 19 samples from patients with T cell acute leukemias (T-ALL) revealed that 5/19 (26%) had low/negative expression of NKAP. Although the sample size is too small for statistical analysis of outcome, this demonstrates that mutations that alter NKAP expression may be a common event in human cancer.

Aberrant expression of NKAP in ovarian cancer may be caused by mutations in its coding sequence. Interestingly, the NKAP gene is on the X chromosome. In females, since each cell contains two copies of the X chromosome, one of the chromosomes is heritably silenced through the process of random X inactivation. Thus, in contrast to autosomally-located genes that are expressed from both copies on paired chromosomes, only one allele of most X-chromosome located genes, including NKAP, is expressed in each female cell. Therefore, in contrast to the ‘two-hit’ requirement for gene inactivation, NKAP function could be disrupted by a single mutation in the expressed copy of the gene. To determine whether ovarian cancer samples with aberrant expression of NKAP have mutations within the coding region, NKAP mRNA was isolated from frozen tumor samples and used to generate cDNA and perform RT-PCR. The cDNAs were sequenced to determine whether mutations that alter the amino acid sequence are present. A panel of 15 ovarian tumor samples was analyzed. One of these, OV1539, was found to have a three nucleotide deletion in the amplified cDNA, which results in the deletion of a serine at position 201. This mutation did not correspond to any known SNP in the database. Therefore, there are, and may be additional, mutations in NKAP in primary ovarian cancer samples.

### Example 2

### Assessing Mutations in NKAP

For those samples that have mutations in NKAP that alter its protein sequence (either truncations or amino acid substitutions), biochemical analyses may be required to determine whether these mutations alter NKAP expression, function, or localization. Assays described elsewhere (Pajerowski et al., Immunity, 30:696-707 (2009)) are used to examine NKAP expression, localization, and function in cell lines. Briefly, the mutations found in ovarian tumor samples are re-created in YFP-tagged NKAP expression constructs. These NKAP mutants are transfected into 293T cells to examine alterations in localization by fluorescent microscopy. Mean fluorescent intensity upon transfection is compared to determine whether there are defects in expression of the mutated polypeptides as compared to wild type NKAP polypeptides. The ability of mutated NKAP to inhibit Notch-driven transcription of a CSL-luciferase reporter upon cotransfection is also examined. The expression/localization of each mutated NKAP should correspond to its expression/localization as originally characterized by IHC. For example, NKAP expression was low in OV1539, which has a deletion of 5201, and this mutation may result in altered expression of NKAP (perhaps through failure to fold properly, which would lead to increased degradation and loss of expression).

### Example 3

### Loss of Copy Number at the NKAP Locus

Consistent with the results that NKAP expression was lost in a subset of ovarian cancers, mining of The NCI Cancer Genome Atlas (TCGA) for alterations in NKAP expression in human malignancies demonstrated that there is a loss of copy number at the NKAP locus in ovarian cancer. The analysis of copy number in ovarian cancer was performed using the Biologically Oriented Repository Architecture (BORA), a database for System biology applications from the Mayo Clinic-IBM collaboration. Of 255 ovarian tumors with copy number variation (CNV) data in TCGA, 136 exhibited evidence of significant copy number loss at the NKAP locus. To account for tumor heterogeneity which attenuates CNV deletion signals, significant CNV loss was defined by greater than three standard deviations below the minimum of the average measured CNV for normal tissue or the value measured in germline DNA for that patient. If the loss of heterozygosity is in the expressed allele of NKAP, then it would be predicted that CNV loss would mirror decreases in NKAP expression levels. log 2CNV vs. log 2expression was analyzed. It was found that there was a correlation with R²=0.19 and p-value of 5.226e-13, indicating a strong correlation between loss of copy number and decreased expression of NKAP. There is an inverse correlation between NKAP expression and expression of the Notch-target gene Deltex1 in ovarian cancer (DTX1 Expression vs. NKAP expression (p-value=4e-5) or vs. NKAP CNV (p-value 9e-3), indicating that loss of NKAP expression leads to increased Notch target expression in ovarian cancer. Therefore, one mechanism of loss of NKAP expression may be loss of copy number at the expressed allele. This can be confirmed by paraffin FISH of ovarian cancer samples in which NKAP expression was shown to be low/negative by IHC. In addition, these sections can be co-stained by Xist FISH (Clemson et al., J. Cell Biol., 132:259-275 (1996)) to identify whether the loss of heterozygosity is in the active or inactive X chromosome. Thus, these studies will confirm whether deletions at the NKAP locus occur in ovarian cancer in those samples with loss of NKAP expression.

### Example 4

### Analysis of NKAP Expression and Clinical Outcomes for Ovarian Cancers

The following was performed to build on to the sample set of Example 1. A larger study with 357 human patients with ovarian cancer were assessed for NKAP expression and classified as having normal NKAP expression (NKAP1), no or low NKAP expression (NKAP=2), or cytoplasimic/mislocalized NKAP expression (NKAP3). Across all morphologies (serous, endometrioid, clear cell, mucinous, or mixed), there were 29 cases (8.1%) with no/low expression (NKAP2) and 17 cases (4.8%) with expression in the cytoplasm/mislocalization group (NKAP3) (Table 1). There was no association between NKAP expression and disease stage (p=0.85). There were some significant differences in NKAP expression across tumor morphologies with cytoplasm/mislocaliziation most common in mucinous (23%) and endometrioid (8%) compared to serous (3%). No/low expression was most common in serous (10%) and clear cell (11%) compared to mucinous (0%) and endometrioid (3%).

TABLE 1
Cancer morphologies and NKAP expression.

| | | | Clear | | | |
|---|---|---|---|---|---|---|
| | Serous | Endometrioid | Cell | Mucinous | Mixed | All |
| Normal | 210 | 54 | 15 | 10 | 21 | 310 |
| NKAP | (87%) | (89%) | (83%) | (77%) | (91%) | (87%) |
| No/Low | 23 | 2 | 2 | 0 | 2 | 29 |
| NKAP | (10%) | (3%) | (11%) | | (9%) | (8%) |
| (“NKAP2”) | | | | | | |
| NKAP mis- | 8 | 5 | 1 | 3 | 0 | 17 |
| localization | (3%) | (8%) | (6%) | (23%) | | (5%) |
| (“NKAP3”) | | | | | | |

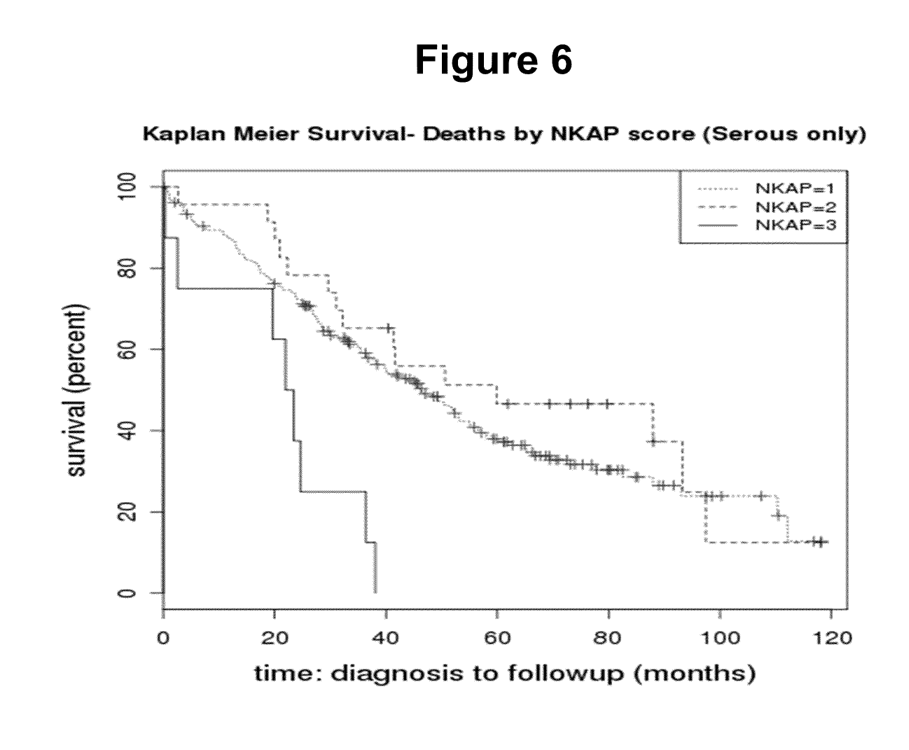

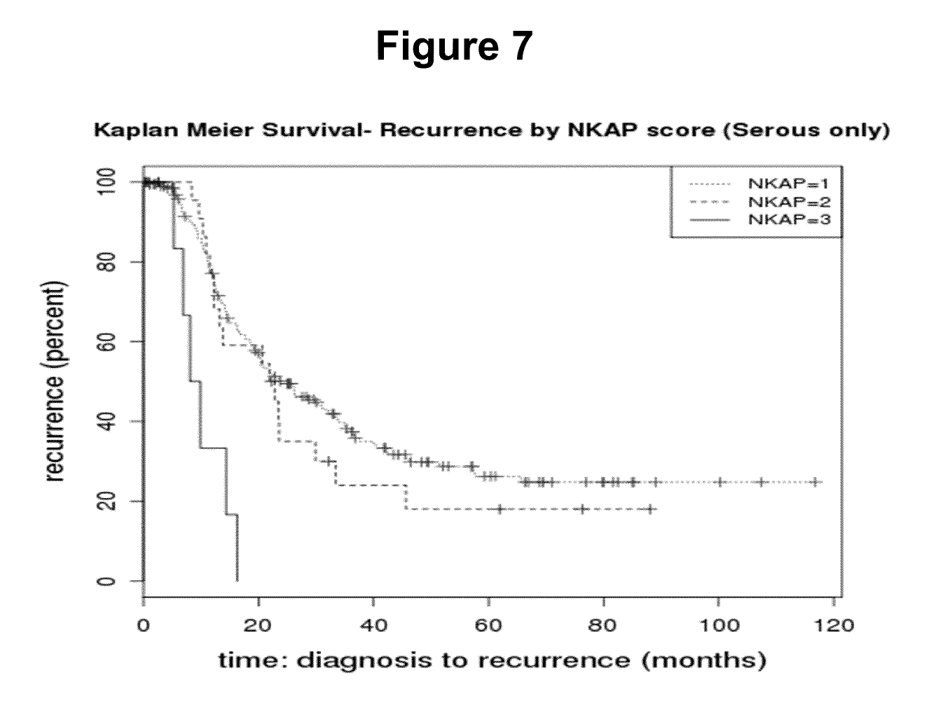

After adjusting for stage and morphology (Table 2), NKAP expression in the cytoplasm/mislocaliziation was significantly associated with poor overall survival (HR=2.19, p=0.01) and time to recurrence (HR=3.59, p=0.0005) compared to patients with normal NKAP expression. These remain significant after subsetting to serous only (HR=3.26, p=0.002; TTR HR=4.65, p=0.0003). There was no significant association between no/low expression and outcome; these patients had similar outcome to patients with normal NKAP expression. For patients within the serous subset, the presence of cytoplasimic/mislocalized NKAP expression (NKAP3) was significantly associated with poor overall survival (FIG. 6) and time to recurrence (FIG. 7).

TABLE 2
NKAP expression and survival.

| Model | | | | | | |
|---|---|---|---|---|---|---|
| Adjust- | | | # | | | |
| ments | Subtype | Variable | deaths | HR | CI | P |
| none | all | NKAP2 | 198 | 1.18 | 0.75-1.87 | 0.46 |
| | | NKAP3 | | 1.41 | 0.79-2.54 | 0.25 |
| stage, | all | NKAP2 | 198 | 1.09 | 0.69-1.73 | 0.71 |
| subtype, | | | | | | |
| age | | | | | | |
| | | NKAP3 | | 2.21 | 1.20-4.09 | 0.012 |
| none | Serous | NKAP2 | 150 | 0.78 | 0.46-1.33 | 0.36 |
| | | NKAP3 | | 3.5 | 1.69-7.23 | 0.0007 |
| stage, age | Serous | NKAP2 | 150 | 0.85 | 0.49-1.46 | 0.55 |
| | | NKAP3 | | 3.35 | 1.62-6.93 | 0.001 |
| none | Endo- | NKAP2 | 23 | 3.63 | 0.83-15.85 | 0.086 |
| | metrioid | | | | | |
| | | NKAP3 | | 1.79 | 0.53-6.11 | 0.35 |
| stage, age | Endo- | NKAP2 | 23 | 0.62 | 0.13-3.12 | 0.57 |
| | metrioid | | | | | |
| | | NKAP3 | | 2.22 | 0.63-7.84 | 0.22 |
| none | Mu- | NKAP2 | 2 | NA | | |
| | cinous | | | | | |
| | | NKAP3 | | NA | | |
| stage, age | Mu- | NKAP2 | 2 | NA | | |
| | cinous | | | | | |
| | | NKAP3 | | NA | | |
| none | Clear | NKAP2 | 10 | 27.4 | 2.38-314.95 | 0.0079 |
| | cell | | | | | |
| | | NKAP3 | | 2.3 | 0.27-19.86 | 0.45 |
| stage, age | Clear | NKAP2 | 10 | 28.87 | 2.32-359.4 | 0.009 |
| | cell | | | | | |
| | | NKAP3 | | 1.33 | 0.15-11.84 | 0.80 |

HR = hazard ratio;
CI = 95% confidence interval;
P = probability

These results demonstrate that NKAP expression is either lost/low (NKAP2) or mislocalized (NKAP3) in a substantial fraction of ovarian cancers. NKAP expression was lost in 29/355 (8%) and mislocalized in 17/355 (5%) in this cohort. Mislocalization of NKAP was a negative prognostic indicator for overall survival across all subtypes (HR: 2.21, CI: 1.20-4.09, p=0.012). In serous subtypes, NKAP mislocalization (NKAP2) was a negative prognostic indicator for overall survival, while NKAP expression loss (NKAP3) trended towards being a positive prognostic indicator (NKAP2: HR: 0.85, CI: 0.49-1.46, p=0.55; NKAP3: HR: 3.35, CI 1.62-6.93, p=0.001). Misexpression of NKAP (NKAP3) was not associated with outcome in endometroid subtype, and there were too few samples with altered NKAP expression to examine outcome for mucinous. In clear cell subtype, loss of NKAP expression (NKAP2) was a strong negative indicator for overall survival, with independent statistical significance (NKAP2: HR: 28.87, CI: 2.32-359.4, p=0.009). Even if Cox regression adjustments were not performed, these results were still highly significant (NKAP2 without Cox modeling: HR: 27.4, CI: 2.38-314.95, p=0.0079).

### Example 5

### NKAP Expression and T-ALL

Since mutations that result in increased Notch signaling contribute to leukemogenesis, it was hypothesized that mutations that lead to loss of NKAP expression or function may also be found in human T-ALL. Several groups have characterized gene expression patterns in T-ALL by microarrays using Affymetrix arrays (HU6800, U95Av2, and U74Av2) that, however, lack NKAP.

NKAP expression was examined in 21 primary T-ALL samples by immunohistochemistry for NKAP expression, using rabbit polyclonal antisera. No reactivity was observed when pre-immune serum was used.

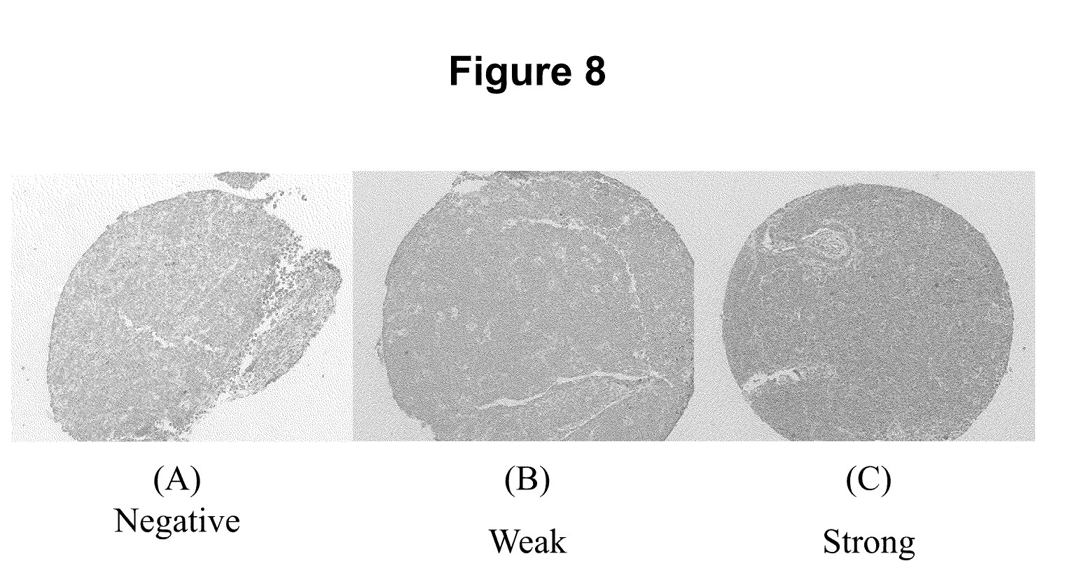

A tissue microarray (TMA) composed of triplicate cores from archived paraffin sections of peripheral (primarily lymph node) biopsies from 20 patients was used. The patients included 12 males and 8 females with a mean age of 27 years (range 8-65 years). After staining for NKAP expression by immunohistochemistry, NKAP expression within the tumor cells within each core was scored for strong, moderate, weak and negative expression of NKAP. One of the sections did not contain any tumor within the core analyzed, and was dropped from further analysis. One patient was represented twice within the array, at initial diagnosis and at relapse. Both of these samples had strong staining for NKAP, so the patient was counted once. 12/19 T-ALL samples had strong NKAP staining, 3/19 had moderate staining, 2/19 had weak staining, and 3/19 were negative for NKAP expression. An example of strong, weak, and negative staining of the core samples for NKAP are shown (FIG. 8). Thus, 26.3% ( 5/19) of human T-ALL samples had significantly lower levels of NKAP expression. A 26% rate of aberrant expression would make NKAP among the most frequently mutated genes in T-ALL.

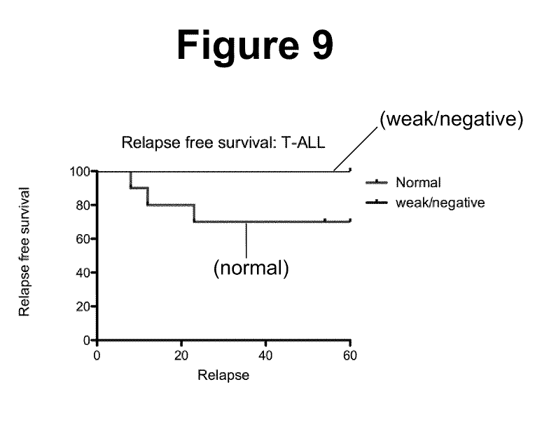

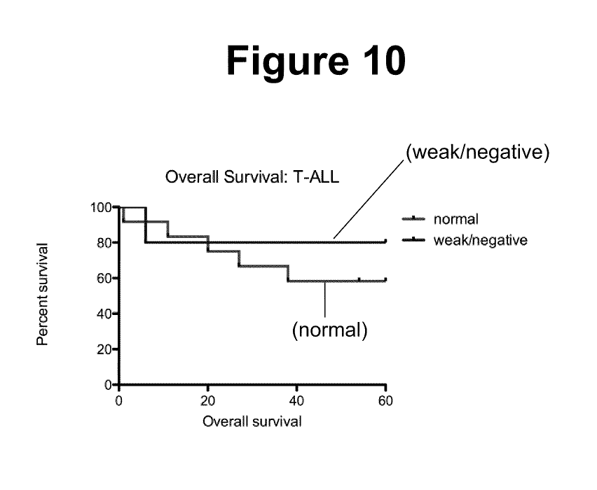

The effects of NKAP deficiency on relapse-free survival and overall survival in this sample set were analyzed. Two patients were lost to follow-up prior to completion of therapy and were deleted from further analysis. Three patients did not achieve remission, and were omitted from the relapse analysis. Kaplan Meier analysis of relapse free survival and overall survival was performed. The data were censored at 60 months, although the average for those without relapse or progression was 99 months. Of the 14 patients who entered remission after initial treatment, 3/10 (30%) patients with strong/moderate NKAP expression relapsed, while 0/4 (100%) patients with weak/negative NKAP staining relapsed (FIG. 9). Overall survival was 58% ( 7/12) in patients with strong/moderate NKAP expression, while it was 80% (⅘) for patients with weak/negative NKAP staining (FIG. 10). One patient with weak NKAP expression progressed and died during bone marrow transplantation, and was included in the overall survival but not in the relapse data since remission was not achieved. Although provocative, because of the small sample size, this data is not yet statistically significant using either the Log-rank (Mantel-Cox) or Gehan-Breslow-Wilcoxon tests. However, it appears that the loss of NKAP is a positive prognostic indicator in T-ALL, which would be consistent with previously published results demonstrating patients with activating mutations in Notch1 have better relapse-free survival and overall survival than patients without mutations in Notch1 (Mansour et al., J. Clin. Oncol., 10:4352-4356 (2009); and Asnafi et al., Blood, 113:3918-3924 (2009)).

### Example 6

### NKAP Expression and Pancreatic Cancer

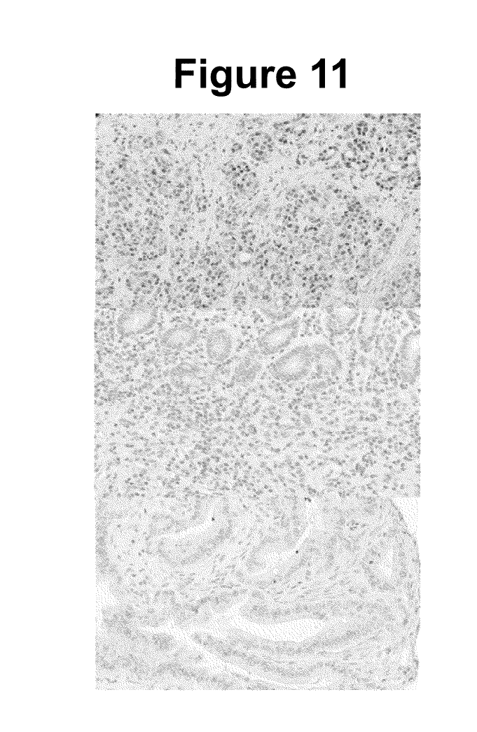

Tissue microarrays were previously generated from surgical samples from curative resections, arising from 41 patients with margin-negative small pancreatic adenocarcinomas (less than 2 cm) and matched with 94 patients with large (greater than 2 cm) pancreatic adenocarcinomas for tumor stage and differentiation (Pongprasobchai et al., Pancreatology, 8:587-592 (2008)). The TMAs were stained for NKAP expression, and scored by a pathologist. Not all patient samples contained tumor in the section examined, and if not, they were dropped from further analysis. Additionally, a TMA generated from patients with pre-cancerous pancreatic lesions (PaNINs) were also examined. Similar to what was found in ovarian cancer and T cell acute leukemias, the expression of NKAP was heterogeneous. In the top panel of FIG. 11 is an example for normal nuclear NKAP staining in a patient's pancreatic adenocarcinoma. In the middle panel is an example of a patient's pancreatic adenocarcinoma with low/negative NKAP expression, and in the bottom panel is an example of a patient's pancreatic adenocarcinoma with mislocalized NKAP expression (all samples were counterstained with hematoxylin). Of 45 pancreatic adenocarcinoma patient tumors examined, 11/45 (24%) expressed normal nuclear expression of NKAP, 25/45 (56%) expressed low or were negative for expression of NKAP, and 9/45 (20%) had cytoplasmic mislocalization of NKAP. The median survival post surgery for patients with pancreatic adenocarcinomas with normal nuclear expression of NKAP was 499 days, as compared to 675 days for patients with low/negative NKAP expression and 521 days for patients with cytoplasmic mislocalization (Table 3). These results are not statistically significant; however, patients with low/negative NKAP expression fared worse than patients with normal NKAP expression with regard to 5 year survival. Of patients with pancreatic adenocarcinomas with normal NKAP expression, only 4/11 (36%) were alive five years post-surgery. Only 4/25 (16%) of patients with pancreatic adenocarcinomas with low/negative NKAP expression were alive five years post-surgery. Therefore, loss of NKAP expression in pancreatic adenocarcinomas decreases the likelihood by two-fold that the patient would survive five years post-surgery.

TABLE 3

| | Normal | | Low/Negative | | Cytoplasmic | | Total | |
|---|---|---|---|---|---|---|---|---|
| Adenocarcinoma Cores | | | | | | | | |
| Survival post Surgery (days) | | | | | | | | |
| N | 11 | | 25 | | 9 | | 45 | |
| Mean (SD) | 1417.7 | (1486.17) | 1030.6 | (980.19) | 1316.8 | (1373.86) | 1182.5 | (1183.15) |
| Median | 499.0 | | 675.0 | | 521.0 | | 541.0 | |
| Q1, Q3 | 404.0, 2591.0 | | 351.0, 1477.0 | | 371.0, 1889.0 | | 377.0, 1587.0 | |
| Range | (325.0-4820.0) | | (121.0-4336.0) | | (13.0-4109.0) | | (13.0-4820.0) | |
| 5-year survival | | | | | | | | |
| Deceased by 5 years | 7 | (63.6%) | 21 | (84%) | 6 | (66.7%) | 34 | (75.6%) |
| Alive at 5 years | 4 | (36.4%) | 4 | (16%) | 3 | (33.3%) | 11 | (24.4%) |
| Pan In Cores | | | | | | | | |
| Survival post Surgery (days) | | | | | | | | |
| N | 9 | | 30 | | 6 | | 45 | |
| Mean (SD) | 877.3 | (754.64) | 1216.2 | (1198.48) | 1471.7 | (1673.86) | 1182.5 | (1183.15) |
| Median | 675.0 | | 531.0 | | 470.0 | | 541.0 | |
| Q1, Q3 | 335.0, 1359.0 | | 371.0, 1889.0 | | 438.0, 2723.0 | | 377.0, 1587.0 | |
| Range | (13.0-2413.0) | | (121.0-4820.0) | | (393.0-4336.0) | | (13.0-4820.0) | |
| 5-year survival | | | | | | | | |
| Deceased by 5 years | 8 | (88.9%) | 22 | (73.3%) | 4 | (66.7%) | 34 | (75.6%) |
| Alive at 5 years | 1 | (11.1%) | 8 | (26.7%) | 2 | (33.3%) | 11 | (24.4%) |

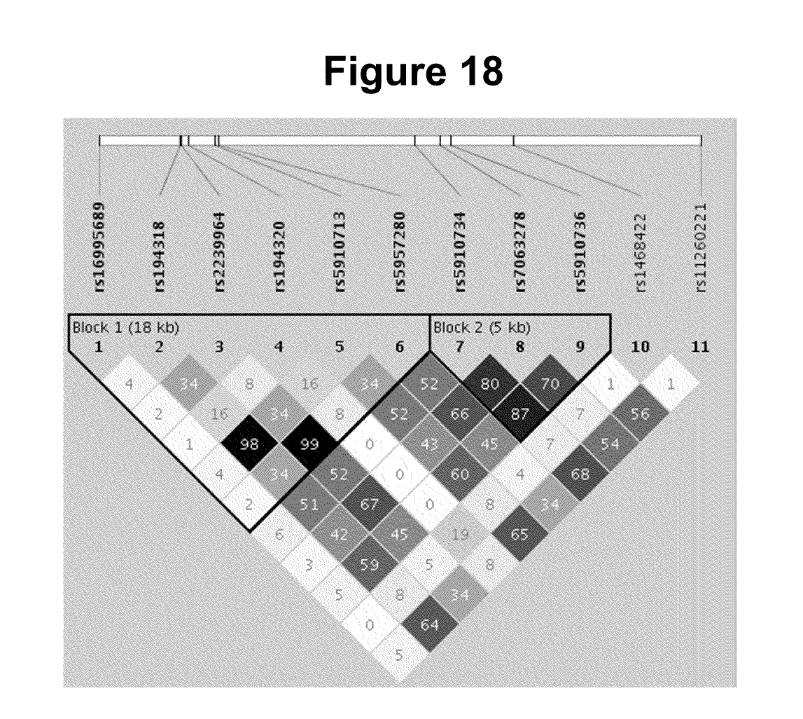

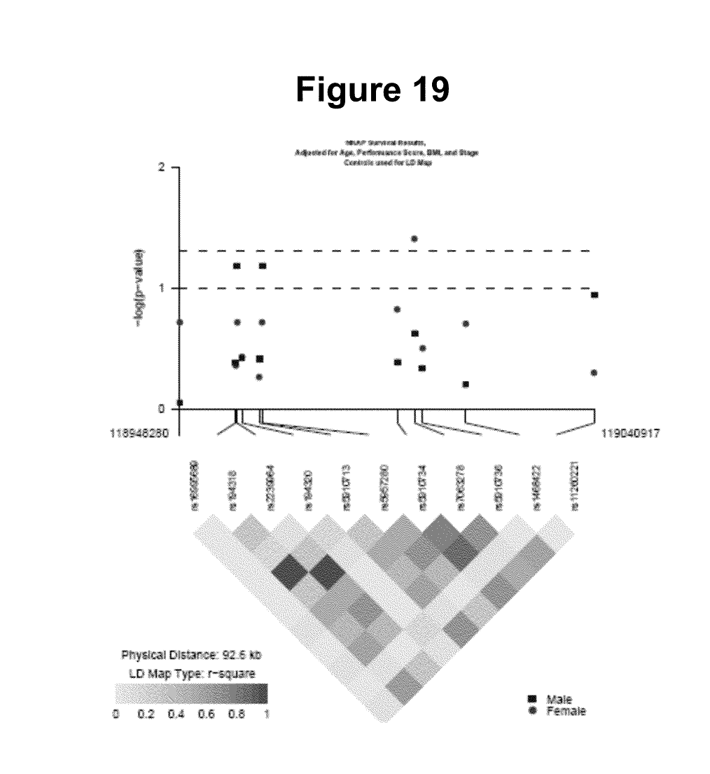

To determine whether there is any association with NKAP and pancreatic adenocarcinomas, 11 SNPs associated with the NKAP locus were examined from a dataset generated for a genome wide association study (GWAS) in pancreatic cancer (PanScan GWAS, Petersen et al., Nat. Genet., 42:224-228 (2010)). There was one SNP (rs7063278) that had a p-value of 0.039 for females in the survival analysis (FIGS. 18 and 19). No other SNPs had p-values <0.05 in any tested analysis. The p-value to claim statistical significance based on a bonferroni correction would be 0.05/11=0.0045. The analysis implicates the NKAP locus as having a role in pancreatic cancer.

NKAP expression level was then analyzed in a larger cohort of patients with pancreatic cancer using the Chari Large and Small TMA set. Descriptive statistics were calculated on the 102 patients with both NKAP expression and survival data available, and correlation between expression and survival were investigated. NKAP expression levels were analyzed using 3 levels (Normal, Low/negative, Cytoplasmic) and also grouped 2 level analyses (Normal vs. Aberrant). In order to handle the multiple observations (1/core) for a given individual the maximum NKAP score for a subject was utilized in the survival analyses. Kaplan Meier Curves were used to visualize the association between NKAP expression and survival. Cox Proportional Hazards Regression models looking at expression alone as well as incorporating adjustment for sex and age at surgery were also considered. Analyses were conducted using SAS 9.1.3. All tests were two-sided, and p-values <0.05 were considered statistically significant.

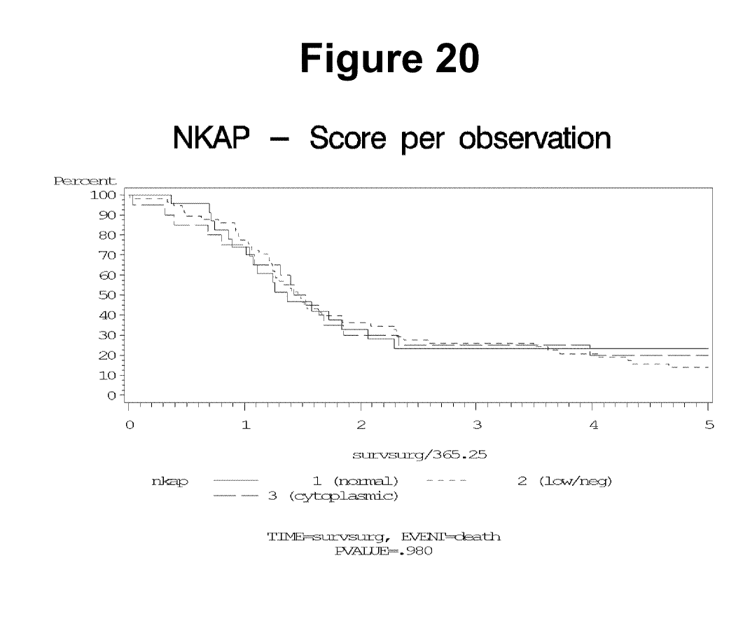

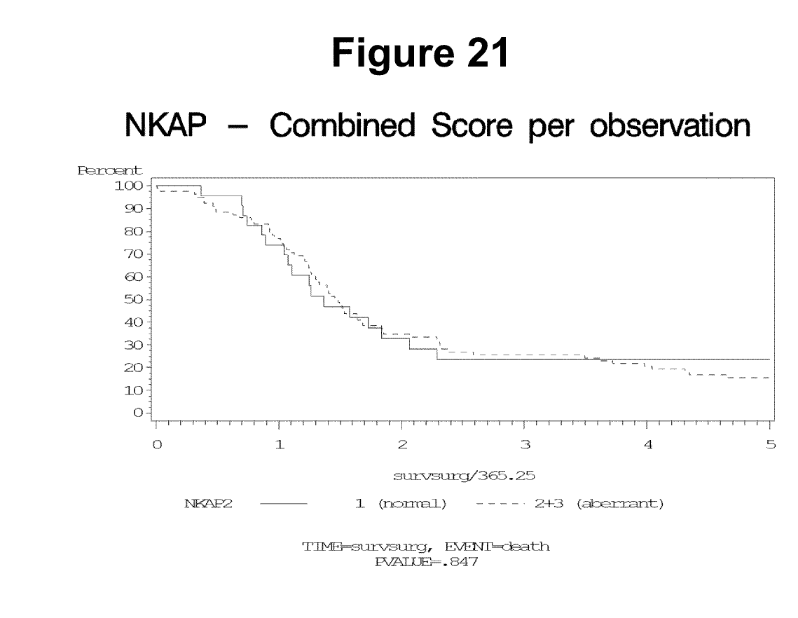

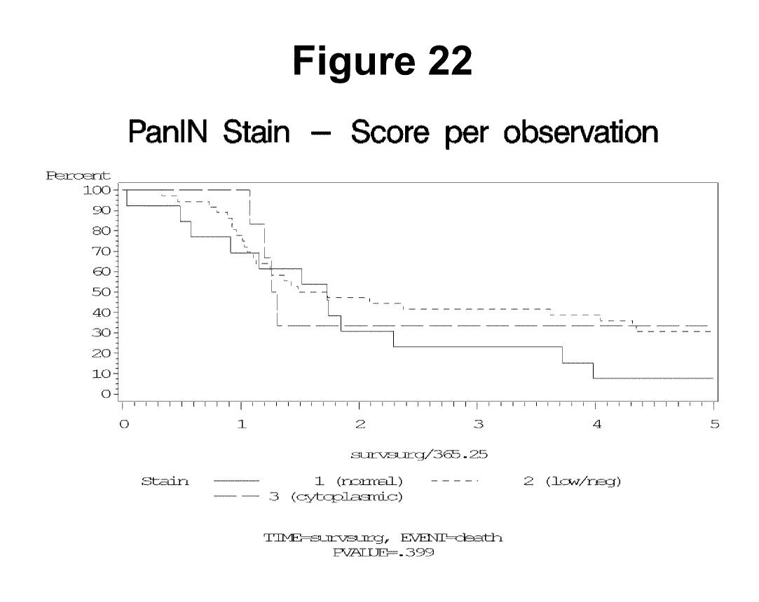

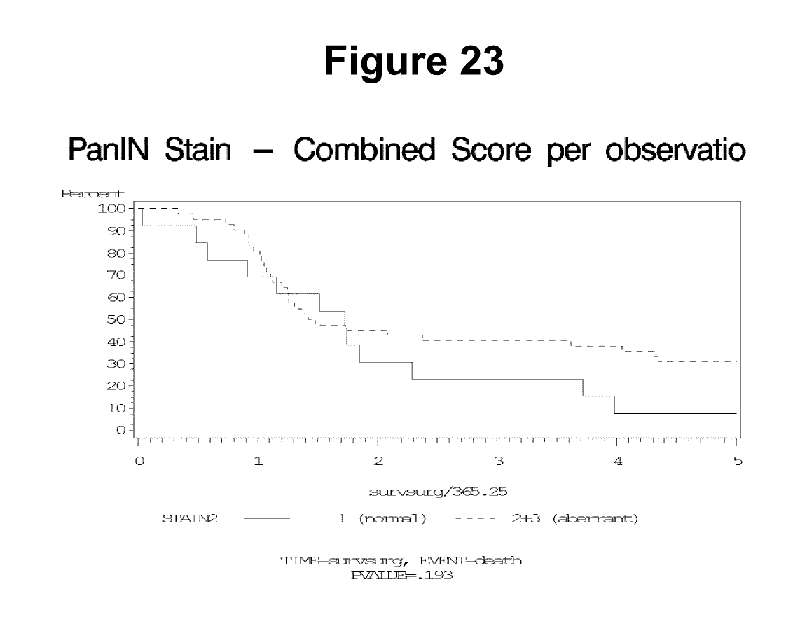

Descriptive statistics are provided in Table 4. Table 5 contains the results of the survival analysis looking at the maximum NKAP expression level within adeno cores for a subject. Table 6 contains the results of the survival analysis looking at the maximum NKAP expression level within PanIN Cores (in adenocarcinoma setting) for a subject. Kaplan Meier survival curves for the adeno cores are provided in FIGS. 20 and 21, and KM curves for the PanIN cores are provided in FIGS. 22 and 23. Based on these results, NKAP expression level does not appear to be associated with survival in this set of 102 patients.

TABLE 4
Descriptive statistics

| Discrete Variable | N | % |
|---|---|---|
| Sex | | |
| Female | 43 | 42.2 |
| Male | 59 | 57.8 |

| | |
|---|---|
| Age at Surgery (mean ± SD) | 63.1 ± 11.5 |
| Vital status | |

| | | |
|---|---|---|
| Alive | 12 | 11.8 |
| Dead | 90 | 88.2 |

| | |
|---|---|
| Follow-up, days (median, Range) | 517 (4, 4820) |

TABLE 5
Cox Proportional Hazards Regression Survival analysis
results-Adeno cores

| | | Me- | Unadjusted | | | Adjusted* | | |
|---|---|---|---|---|---|---|---|---|
| | | dian | Haz- | | | Haz- | | |
| | N/ | Sur- | ard | 95% | p- | ard | 95% | p- |
| | Events | vival | Ratio | CI | value | Ratio | CI | value |
| NKAP | | | | | 0.98 | | | 0.98 |
| Expres- | | | | | | | | |
| sion | | | | | | | | |
| Normal | 23/20 | 499 | 1.00 | — | | 1.00 | — | |
| Low/ | 59/52 | 541 | 1.06 | 0.62, | | 0.95 | 0.55, | |
| Negative | | | | 1.79 | | | 1.62 | |
| Cyto- | 20/18 | 540 | 1.04 | 0.54, | | 1.00 | 0.52, | |
| plasmic | | | | 1.97 | | | 1.89 | |
| NKAP | | | | | 0.85 | | | 0.88 |
| Expres- | | | | | | | | |
| sion | | | | | | | | |
| Normal | 23/20 | 499 | 1.00 | — | | 1.00 | — | |
| Aberrant | 79/70 | 541 | 1.05 | 0.63, | | 0.96 | 0.57, | |
| | | | | 1.75 | | | 1.61 | |

*Adjusted for Sex and Age at Surgery

TABLE 6
Cox Proportional Hazards Regression Survival analysis
results-PanIN cores in Adeno Setting

| | | Me- | Unadjusted | | | Adjusted* | | |
|---|---|---|---|---|---|---|---|---|
| | | dian | Haz- | | | Haz- | | |
| | N/ | Sur- | ard | 95% | p- | ard | 95% | p- |
| | Events | vival | Ratio | CI | value | Ratio | CI | value |
| NKAP | | | | | 0.405 | | | 0.284 |
| Expres- | | | | | | | | |
| sion | | | | | | | | |
| Normal | 13/12 | 632 | 1.00 | — | | 1.00 | — | |
| Low/ | 36/32 | 587 | 0.65 | 0.33, | | 0.60 | 0.30, | |
| Negative | | | | 1.30 | | | 1.20 | |
| Cyto- | 6/5 | 470 | 0.54 | 0.18, | | 0.49 | 0.16, | |
| plasmic | | | | 1.59 | | | 1.46 | |
| NKAP | | | | | 0.196 | | | 0.123 |
| Expres- | | | | | | | | |
| sion | | | | | | | | |
| Normal | 13/12 | 632 | 1.00 | — | | 1.00 | — | |
| Aberrant | 42/37 | 531 | 0.64 | 0.32, | | 0.58 | 0.29, | |
| | | | | 1.25 | | | 1.15 | |

*Adjusted for Sex and Age at Surgery

In the pancreas, NKAP expression is highly expressed in acinar tissue as well as islets. Using antiserum, NKAP expression was examined in the PaNIN/PDAC tissue microarray (TMA), which has been described elsewhere (Pongprasobchai et al., Pancreatology, 8:587-592 (2008)). The stained TMA was scored. Of the pancreatic adenocarcinomas from 102 patients examined, 23/102 (22.5%) had normal nuclear expression of NKAP, 59/102 (57.8%) had low/negative expression of NKAP, and 20/102 (19.6%) had aberrant localization of NKAP to the cytoplasm rather than the nucleus (see also Table 5).

Examining defects in NKAP expression and localization together, 79/102 (77.5%) patients had alterations in NKAP. By comparison, this would make NKAP the third most frequently altered or misexpressed gene in human pancreatic adenocarcinomas, after K-Ras and p16 Ink4a (see Table 7 for comparison). Alteration in NKAP expression was not predictive of outcome, however, this does not exclude that such alterations may play an important role in the development or maintenance of pancreatic cancer.

Altered expression of NKAP was an early event in the development of pancreatic cancer and was observed in PanIN1 lesions. If changes in NKAP expression are directly involved in the generation of pancreatic cancer, one would expect to also find alterations in NKAP in pre-cancerous PanIN (pancreatic intraepithelial neoplasia) lesions. In the spectrum of genetic alterations present in PanIN lesions, there is a hierarchy of expression amongst commonly mutated genes in pancreatic cancer. Activating mutations in K-Ras are found in greater than 95% of pancreatic cancers and PanIN lesions, starting at the earliest PanIN1 stage. Therefore, mutation of K-Ras is considered an initiating event. Consistent with this, mice which express activated Kras only in pancreatic tissue (pdx1-cre K-Ras G12D) spontaneously develop PanINs, but progression to PDAC is rare and occurs only with a long latency, indicating additional mutations are required (Hingorani et al., Cancer Cell, 4:437-450 (2003)). In contrast, alterations in p53 or p16 Ink4a are not present in PanIN1 lesions, but are found in later PanIN2, PanIN3 and PDACs, indicating that they function in progression rather than initiation. This was demonstrated experimentally as mutations in p53 or p16 Ink4a alone do not lead to PanIN or PDAC in mice, however, such mutations do drive the development of PDACs in combination with activating mutations in K-Ras G12D (Hingorani et al., Cancer Cell, 7:469-483 (2005); and Aguirre et al., Genes Dev., 17:3112-3126 (2003)). Therefore, examination of alterations in NKAP expression in human PanIN lesions could indicate whether loss of NKAP may function as either an initiator or driver of oncogenesis.

Examination of NKAP alterations in human PanINs demonstrated that aberrant expression of NKAP (either low/negative or mislocalized to the cytoplasm) is observed equivalently in PanIN lesions across grades. NKAP expression was altered in 13/20 (65%) of PanIN1, 17/18 (94.5%) of PanIN2, and 12/16 (75%) of PanIN3. Therefore, NKAP expression was altered at the earliest stage of PanIN development, indicating that it may play a role in the initiation of pancreatic neoplasms. Only two other genetic modifications have been identified thus far at the PanIN1 stage: K-Ras and p16 Ink4a (see Table 7).

TABLE 7
Common alterations in human PanIN and PDACs.

| Altered | | | | |
|---|---|---|---|---|
| gene/expression | PDAC | PanIN1 | PanIN2 | PanIN3 |
| KRas | 95-100%^{a} | 24-30%^{b} | 45-83%^{b} | 56-94%^{b} |
| CDKN2a (p16 | 82-98%^{c} | 5-44.4%^{d} | 16-80%^{d} | 40-100%^{d} |
| Ink4a) | | | | |
| NKAP | 77.5% | 65% | 94% | 75% |
| Trp53 (p53) | 70-83%^{e} | 0-5%^{f} | 20-46.7^{f} | 53-88.9^{f} |
| Smad4/Dpn4 | 55-66%^{g} | 0%^{h} | 0%^{h} | 13-31%^{h} |
| BRCA2 | 5-10%ⁱ | 0%^{j} | ND | 100% (1/1 sample |
| | | | | examined)^{j} |

^{a}Almoguera et al., Cell, 53: 549-554 (1988) and Tada et al., Gastroenterology, 110: 227-231 (1996).
^{b}Luttges et al., Pancreas, 27: e57-62 (2003) and Sugio et al., Int. J. Pancreatol., 21: 205-217 (1997).
^{c}Caldas et al., Nat. Genet., 8: 27-32 (1994) and Schutte et al., Cancer Res., 57: 3126-3130 (1997).
^{d}Abe et al., Pancreas, 34: 85-91 (2007); Wilentz et al., Cancer Res., 58: 4740-4744 (1998); and Rosty et al., Am. J. Clin. Pathol., 27: 1495-1501 (2003).
^{e}Redston et al., Cancer Res., 54: 3025-3033 (1994) and Biankin et al., Am. J. Clin. Pathol., 28: 1184-1192 (2004).
^{f}Abe et al., Pancreas, 34: 85-91 (2007) and Biankin et al., Am. J. Clin. Pathol., 28: 1184-1192 (2004).
^{g}Biankin et al., Am. J. Clin. Pathol., 28: 1184-1192 (2004) and Iacobuzio-Donahue et al., Am. J. Clin. Pathol., 157: 755-761 (2000).
^{h}Biankin et al., Am. J. Clin. Pathol., 28: 1184-1192 (2004) and Wilentz et al., Cancer Res., 60: 2002-2006 (2000).
ⁱGoggins et al., Cancer Res., 56: 5360-5364 (1996) and Ozcelik et al., Nat. Genet., 16: 17-18 (1997)).
^{j}Goggins et al., Am. J. Pathol., 156: 1767-1771 (2000).

### Example 7

### Pdx1-Cre NKAP Conditional Knockout Mice Spontaneously Develop Mucinous Cystic Neoplasms (MCNs)

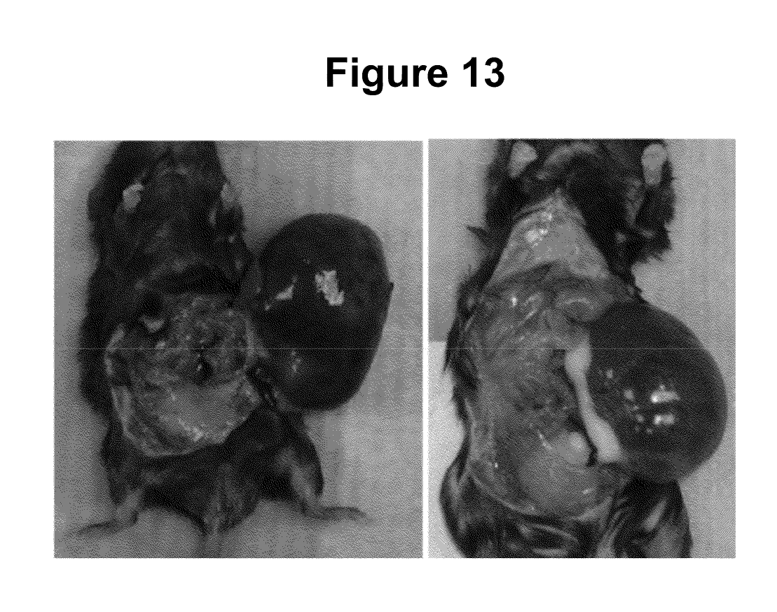

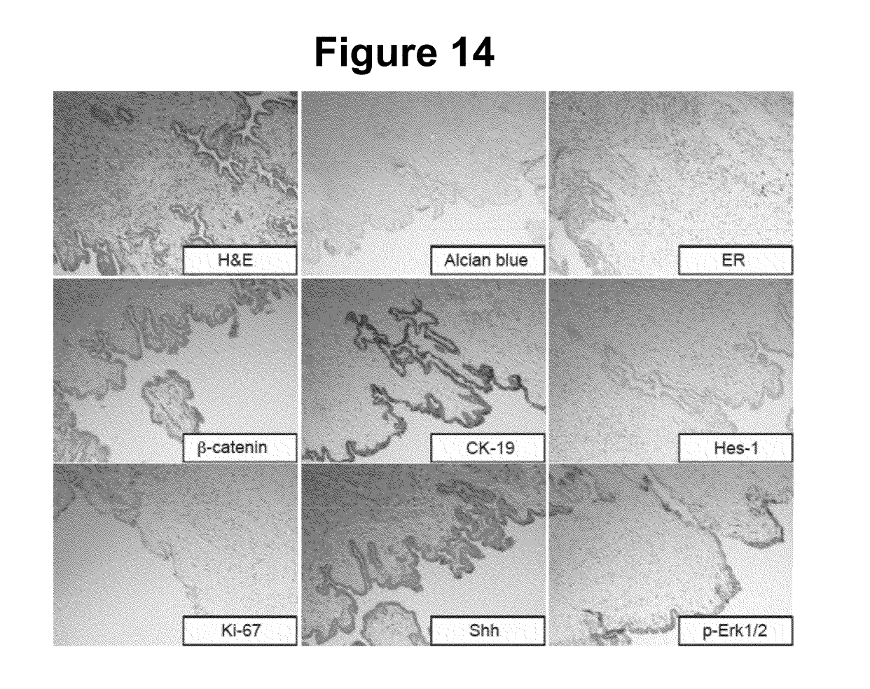

To understand whether the loss of NKAP expression actively contributes to the development of pancreatic cancers in mice, pancreatic specific NKAP conditional knockout mice (pdx1-cre NKAP cKO) were generated. Pdx1-cre NKAP cKO mice were born at the expected Mendelian frequency and were fertile. Glucose levels were normal in pdx1-cre NKAP cKO mice through one year of age, indicating that NKAP is not required for pancreatic development or normal function. Initial examination of pancreatic histology at six months did not reveal any abnormalities, such as PaNIN lesions. Five male Pdx1-cre NKAP cKO were set aside to age. Male mice were chosen, as NKAP is on the X chromosome and therefore male mice contain a single floxed allele of NKAP. So far, two of these mice were euthanized at 12 months and 14 months of age due to abdominal distension (no wild-type littermates have been euthanized to date). In each of these mice, examination of the abdominal cavity revealed a mucinous cyst (FIG. 13). Using the guidelines for IHC established from the NCI/University of Pennsylvania workshop in 2004 (Hruban et al., Cancer Res., 66:95-106 (2006)), the cyst was examined for expression of a panel of markers/stains including H&E, alcian blue, pdx1, sonic hedgehog (Shh), β-catenin, insulin, Hes1, phospho-Erk1/2, cytokeratin-19 (CK-19), estrogen receptor (ER), progesterone receptor (PR), Ki67, synaptophysin and amylase (FIG. 14). Staining with CK-19 confirmed the ductal origin of cells lining the cyst, and alcian blue demonstrated the presence of mucins. These cells also uniformly expressed high levels of Shh and phospho-Erk1/2, which may be indicative of spontaneous activation of K-Ras. β-catenin was also expressed. Hes1 and Ki67 was expressed, albeit at a lower levels and not in all cells. MCN are differentiated from IPMN based on the presence of “ovarian type stroma” characterized by “wavy” nuclei that often, although not always, express ER and PR (Reddy et al., Clin. Gastroenterol. Hepatol., 2:1026-1031 (2004)). As shown in FIG. 14, some of the stromal nuclei were positive for ER, indicating that these cysts may represent MCN. Thus, in the absence of any other genetic manipulation, the conditional ablation of NKAP alone leads to the development of mucinous cysts resembling MCNs.

### Example 8

### Combination of NKAP Deficiency with Oncogenic KRas G12D Drives the Development of PDACs

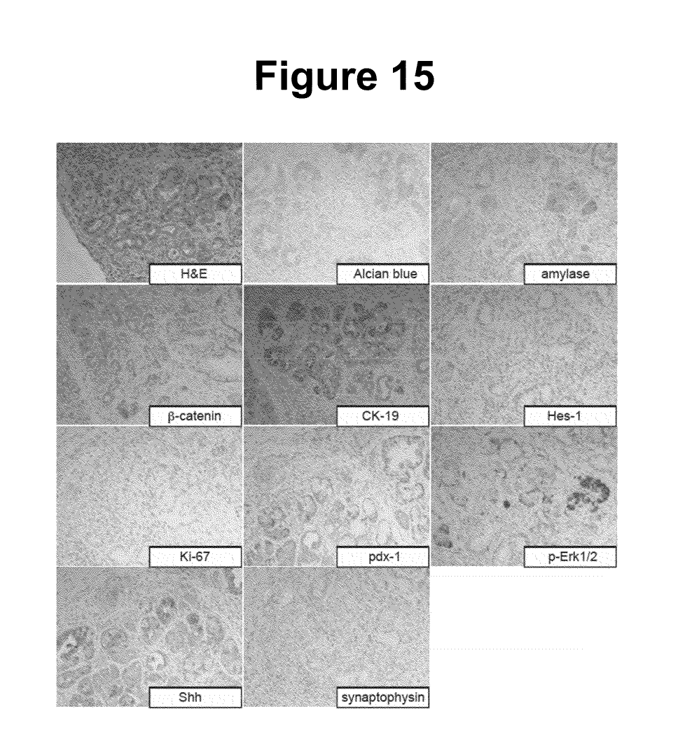

To determine whether loss of NKAP could synergize with oncogenic K-Ras to drive the development of pancreatic cancer, Pdx1-cre NKAP cKO mice were interbred with those with conditional expression of the K-Ras G12D oncogene (“LSL K-Ras G12D”, Tuveson et al., Cancer Cell, 5:375-387 (2004)). As shown in Table 8, pdx1-cre LSL K-Ras G12D developed the spectrum of PanIN lesions over time, and developed PDAC with a low frequency and a long latency. However, oncogenic K-Ras synergizes with loss of tumor suppressors such as p16 Ink4a to drive the development of PDAC with high frequency and short latency. Therefore, it was hypothesized that the loss of NKAP could synergize with oncogenic K-Ras to drive the development of pancreatic adenocarcinomas. Preliminarily, an initial set of pdx1-cre LSL K-Ras G12D and pdx1-cre LSL K-Ras G12D/NKAP “heterozygous” and conditional knockout mice were sacrificed at seven months of age. Since NKAP is located on the X chromosome and undergoes random X inactivation, “het” mice are actually natural chimeras containing a mix of NKAP-expressing and NKAP conditionally deleted cells. The pancreata were fixed in formalin, stained with hematoxylin/eosin, and examined. Consistent with published results, the pancreas from a seven month old pdx1-cre LSL K-Ras G12D mouse contained PanIN1 and PanIN2 lesions. However, the pancreata from pdx1-cre LSL K-Ras G12D/NKAP heterozygous and conditional knockout mice exhibited a complex architecture, with a higher grade of dysplasia and prominent evidence of desmoplasia, with buds of epithelium that arise from PanIN lesions and invade the local tissue, consistent with local invasive ductal adenocarcinoma (FIG. 15).

TABLE 8
Murine models of pancreatic cancer.

| | Pancreatic | |
|---|---|---|
| | abnormalities | |
| | (PanIN or PDAC | Synergize with K-Ras |
| Mouse | development) | G12D |
| Pdx1-cre | PanIN1 until | N/A |
| LSL K-Ras G12D | 6 months; PanIN2 | |
| | development between | |
| | 7-10 months; few | |
| | PanIN3 at 9 months of | |
| | age; rare spontaneous | |
| | progression to PDAC | |
| | after a long latency | |
| | with a median survival | |
| | of 16 months (a) | |
| Pdx1-cre | None (c) | PDAC by 11 weeks (c), |
| p16Ink4a/p19Arf | | average latency of 8.5 |
| cKO | | weeks (d) |
| Pdx1-cre p53 | None (b) | PDAC median survival of |
| R172H | | 5 months with none |
| | | surviving one year (b) |
| Pdx1-cre p53 cKO | None (d) | PDAC by 8 weeks (d) |
| p16 Ink4a KO | None (d) | PDAC with average |
| | | latency of 18 wks (d) |
| Pdx1-cre | None (e, f) | IPMN between 6 and 33 |
| Dpn4/Smad4 cKO | | weeks (e); IPMN between |
| | | 8 and 24 weeks (f) |
| p48-cre | None (g) | MCN with median |
| Dpn4/Smad4 cKO | | survival of 8 months (g) |
| Brca2 Tr/Δ11 | None (h) | PDAC within 110 days (h) |
| Pdx1-cre | None (i) | Inhibition of PDAC |
| Brca2 Δ11/Δ11 | | progression due to |
| | | increased genomic |
| | | instability and apoptosis (i) |

(a) = Hingorani et al., Cancer Cell., 4: 437-450 (2003).
(b) = Hingorani et al., Cancer Cell, 7: 469-483 (2005).
(c) = Aguirre et al., Genes Dev., 17: 3112-3126 (2003).
(d) = Bardeesy et al., Proc. Natl. Acad. Sci. USA, 103: 5947-5952 (2006).
(e) = Kojima et al., Cancer Res., 67: 8121-8130 (2007).
(f) = Bardeesy et al., Genes Dev., 20: 3130-3146 (2006).
(g) = Izeradjene et al., Cancer Cell, 11: 229-243 (2007).
(h) = Skoulidis et al., Cancer Cell, 18: 499-509 (2008).
(i) = Rowley et al., Gastroenterology, 140(4): 1303-1313 (2011).

These PDACs were ductal in origin as shown by expression of CK-19 and staining with Alcian Blue to detect mucins. These lesions expressed amylase, but not insulin or synaptophysin (FIG. 15), demonstrating their exocrine acinar origin. As would be expected due to the engineered mutations in K-Ras and NKAP, the PDACs had constitutive activation of Erk (phospho-Erk expression) and Shh as well as expression of Hes-1, which is regulated by Notch activation. Upregulation of β-catenin was also observed. This desmoplastic response is characteristic of the development of PDAC in humans, although little or no desmoplasia has been observed in other murine genetically engineered models of pancreatic cancer (Hruban et al., Cancer Res., 66:95-106 (2006)). Therefore, within the context of an initial examination of seven month old mice, the loss of NKAP synergized with K-Ras G12D to drive the development of locally invasive adenocarcinoma.

### Example 9

### NKAP Expression and Glioblastomas

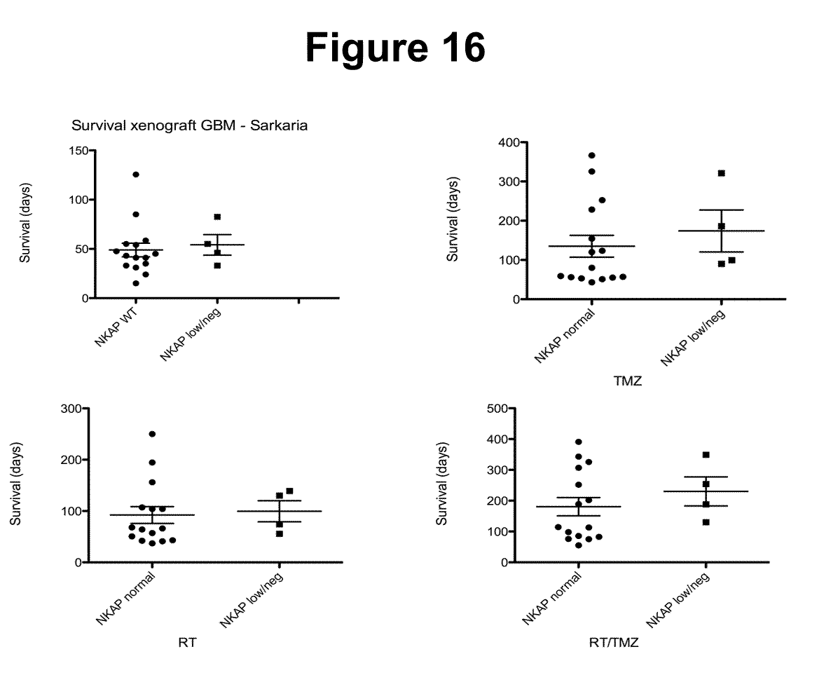

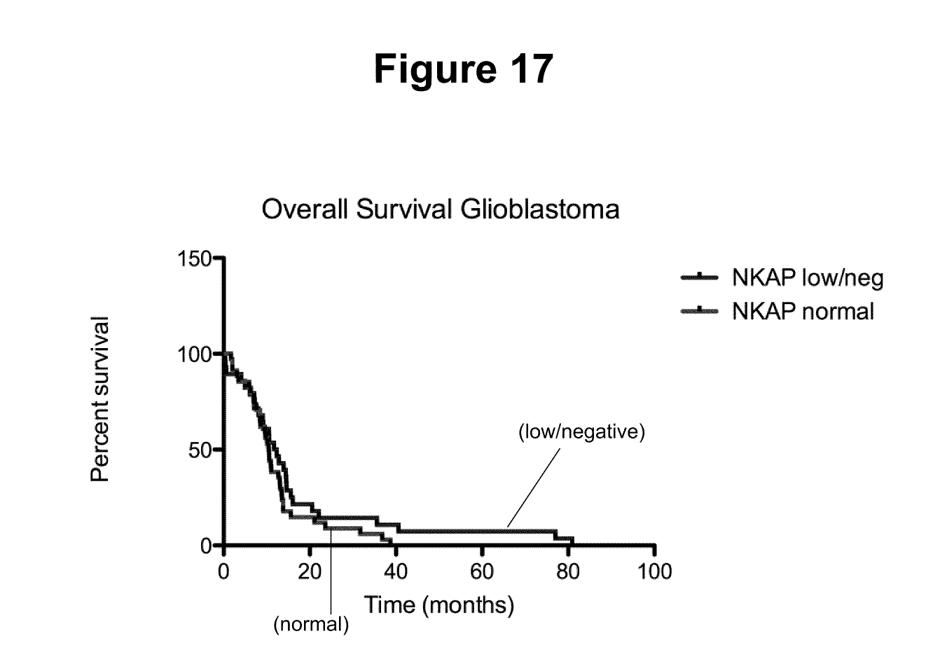

Tissue microarrays previously generated from patients with glioblastoma were analyzed for NKAP expression by immunohistochemistry, and counterstained with hematoxylin (as described above for analysis of NKAP expression in ovarian cancer, T cell leukemia and pancreatic cancer). The TMAs were read by a pathologist. Of the 75 primary patient samples examined, 45/75 (60%) had normal nuclear expression of NKAP, while 30/75 (40%) has low/negative expression of NKAP. No patient samples had cytoplasmic mislocalization of NKAP. Similar results were obtained when NKAP expression was examined in human glioblastoma xenograft tissue microarray, in which primary patient glioblastomas were grown in a mouse xenograft. In tumors arising in this model, 18/24 (75%) had normal nuclear NKAP expression, while 6/24 (25%) had decreased (low/negative) expression of NKAP. Similar to the primary glioblastomas, no glioblastomas from the xenograft model had cytoplasmic mislocalization of NKAP. An example of NKAP staining in glioblastomas is shown in FIG. 16. In the left panel, NKAP expression in normal white matter is shown (FIG. 16). NKAP is strongly expressed in the nucleus of glial cells (the cells from which glioblastomas arise), but not in neurons or in blood vessels. The middle panel shows an example of a glioblastoma with a significant proportion of tumor cells lacking NKAP expression (FIG. 16). The right panel shows an example of a glioblastoma (upper left portion of the section, in the bottom right portion of the section is normal brain tissue) with normal nuclear expression of NKAP (FIG. 16). In the primary patient samples examined, decreased NKAP expression did not affect overall survival (FIG. 17). Interestingly, of those patients with low/negative expression of NKAP, 6.9% were stage 3 ( 2/29) and 93.1% ( 27/29) were stage 4. Of the normal NKAP group, 20.45% ( 9/44) were stage 3, and 79.55% ( 35/44) were stage 4. Staging information at diagnosis was not available for all patients, and therefore the numbers examined were less than that stained for on the tissue microarray. Therefore, decreased NKAP expression appears to correlate with a higher grade, and thus, a more aggressive and invasive disease.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for identifying a mammal as having cancer, wherein said method comprises:
(a) detecting the presence of cells having aberrant NKAP polypeptide expression within a tissue sample from said mammal, and
(b) classifying said mammal as having said cancer based at least in part of the presence of said cells.

2. The method of claim 1, wherein said mammal is a human.

3. The method of claim 1, wherein said cells are ovarian, pancreatic, neuronal, or immune cells.

4. The method of claim 1, wherein said aberrant NKAP polypeptide expression is reduced NKAP polypeptide expression.

5. The method of claim 1, wherein said aberrant NKAP polypeptide expression is cytoplasmic expression of said NKAP polypeptide.

6. A method for assessing a cancer patient's risk for cancer relapse, wherein said method comprises:
(a) determining whether or not a tissue sample from said patient comprises cells having aberrant NKAP polypeptide expression,
(b) classifying said patient as having an increased likelihood for cancer relapse if said cells are present within said sample, and
(c) classifying said patient as not having an increased likelihood for cancer relapse if said cells are absent from said sample.

7. The method of claim 6, wherein said patient is a human patient.

8. The method of claim 6, wherein said patient is a stage 4 ovarian cancer patient.

9. The method of claim 6, wherein said cells are ovarian cells.

10. The method of claim 6, wherein said aberrant NKAP polypeptide expression is reduced NKAP polypeptide expression.

11. The method of claim 6, wherein said aberrant NKAP polypeptide expression is cytoplasmic expression of said NKAP polypeptide.

12. A method for assessing a cancer patient's likelihood for prolonged cancer survival, wherein said method comprises:
(a) determining whether or not a tissue sample from said patient comprises cells having aberrant NKAP polypeptide expression,
(b) classifying said patient as having a reduced likelihood for prolonged cancer survival if said cells are present within said sample, and
(c) classifying said patient as having an increased likelihood for prolonged cancer survival if said cells are absent from said sample.

13. The method of claim 12, wherein said patient is a human patient.

14. The method of claim 12, wherein said patient is a stage 4 ovarian cancer patient.

15. The method of claim 12, wherein said cells are ovarian cells.

16. The method of claim 12, wherein said aberrant NKAP polypeptide expression is reduced NKAP polypeptide expression.

17. The method of claim 12, wherein said aberrant NKAP polypeptide expression is cytoplasmic expression of said NKAP polypeptide.

18. A method for assessing a cancer patient's likelihood for responding to a gamma-secretase inhibitor cancer treatment, wherein said method comprises:
(a) determining whether or not a tissue sample from said patient comprises cells having aberrant NKAP polypeptide expression,
(b) classifying said patient as having a reduced likelihood of responding to said gamma-secretase inhibitor cancer treatment if said cells are present within said sample, and
(c) classifying said patient as being likely to respond to said gamma-secretase inhibitor cancer treatment if said cells are absent from said sample.

19. The method of claim 18, wherein said cancer patient is a human patient.

20. The method of claim 18, wherein said cancer patient is a patient diagnosed with ovarian cancer, pancreatic cancer, leukemia, glioblastoma, T cell lymphomas, or colon adenocarcinomas.

21. The method of claim 18, wherein said cells are ovarian, pancreatic, neuronal, or immune cells.

22. The method of claim 18, wherein said aberrant NKAP polypeptide expression is reduced NKAP polypeptide expression.

23. The method of claim 18, wherein said aberrant NKAP polypeptide expression is cytoplasmic expression of said NKAP polypeptide.
